(19) United States
(12) Patent Application Publication
(10) Pub. No.: US 2013/0079723 A1
(43) Pub. Date: Mar. 28, 2013

(54) MEDICAL SECUREMENT DEVICE WITH TIMER

(71) Applicants: Rafael V. Andino, Grayson, GA (US); Chris Brooks, Glen Head, NY (US)

(72) Inventors: Rafael V. Andino, Grayson, GA (US); Chris Brooks, Glen Head, NY (US)

(73) Assignee: Venetec International, Inc., Covington, GA (US)

(21) Appl. No.: 13/498,117

(22) PCT Filed: Oct. 6, 2010
(86) PCT No.: PCT/US10/51659

§ 371 Date: Dec. 10, 2012

Related U.S. Application Data
(60) Provisional application No. 61/249,222, filed on Oct. 6, 2009.

(51) Int. Cl. A61M 25/02

(52) U.S. Cl. CPC A61M 25/02
USPC 604/180; 604/174

(57) ABSTRACT

A medical article, such as an anchor pad for stabilizing a medical line and/or a medical line securement device to a patient's skin, is provided with a timer. The timer can measure and provide an indication of elapsed time. The timer can be flexible or rigid, and can be disposed directly on the anchor pad, on a flexible or rigid substrate disposed on the anchor pad, or on a retainer supported by the anchor pad. The anchor pad with integral timer provides an easy-to-use visual indicator of elapsed time since placement of the medical line, offering a reliable way to signal when the medical line should be re-sited.

### CROSS-REFERENCE TO RELATED APPLICATIONS

This application claims priority under 35 U.S.C. §119(e) to U.S. Provisional Application No. 61/249,222, filed Oct. 6, 2009, entitled “Anchor Pad with In-Use Timer,” the disclosure of which is hereby incorporated by reference in its entirety.

### BACKGROUND

1. Field of the Invention

This invention relates to a securement device which is attached to a patient's skin and includes a timer. For example, the securement device can be an anchor pad. The anchor pad can directly stabilize a medical line or device relative to the skin of a patient or can stabilize the medical line or device indirectly via a retainer. The timer can measure elapsed time, intervals, etc.

2. Description of the Related Art

It is common in the treatment of patients to utilize catheters to introduce fluids and medications directly into the patient or to withdraw fluids from the patient. Often, it becomes desirable to maintain such catheterization over an extended period of time during the treatment of a patient. In order to keep the catheter or other medical line properly positioned for the duration of treatment, the catheter or medical line can be secured to the patient in a variety of ways. The catheter or medical line may be secured to the patient for an extended period of time. Accordingly, it may be advantageous to locally monitor, for example, the duration of treatment.

### SUMMARY OF THE INVENTION

The devices and methods of the present invention have several features, no single one of which is solely responsible for its desirable attributes. Without limiting the scope of this invention as expressed by the claims which follow, its more prominent features will now be discussed briefly. After considering this discussion, and particularly after reading the section entitled “Detailed Description of Certain Embodiments,” one will understand how the features of this invention provide several advantages over existing medical devices.

An aspect of the invention includes an anchor pad for securing to a patient. The anchor pad includes a timer.

Another aspect of the invention includes a securement system for securing a medical article to the skin of a patient. The securement system comprises an anchor pad having a lower surface with at least a portion of the lower surface being covered by an adhesive. The securement system further includes a retainer supported by the anchor pad, the retainer being configured to receive at least a portion of the medical article. The securement system further includes a timer supported by the securement system.

Another aspect of the invention is directed to a method of indicating dwell time of a medical line in a patient. The method includes inserting the medical line at an insertion site in the skin of the patient, securing an anchor pad to the skin of the patient near the insertion site, the anchor pad comprising a timer configured to provide an indication of elapsed time, and activating the timer.

### BRIEF DESCRIPTION OF THE DRAWINGS

FIG. 1A is a perspective view of a securement device, such as an anchor pad, that includes a timer according to a preferred embodiment of the present invention.

FIG. 1B is a perspective view of the anchor pad of FIG. 1A, shown securing an exemplary medical article.

FIG. 2A is a top view of a portion of an anchor pad according to another embodiment, including a chemically-reactive timer with a removable protective layer.

FIG. 2B is a top view the anchor pad of FIG. 2A, with the protective layer removed.

FIG. 2C is a side view of the anchor pad of FIG. 2B.

FIG. 3 is a top view of a portion of an anchor pad according to a further embodiment, including a battery-powered timer.

FIG. 4 shows a chemically-activated timer according to a further embodiment.

FIG. 5 illustrates the chemically-activated timer from FIG. 4 secured to a patient via an anchor pad.

### DETAILED DESCRIPTION OF CERTAIN EMBODIMENTS

The following description and the accompanying figures, which describe and show the preferred embodiments, are made to demonstrate several possible configurations that a securement device can take to include various aspects and features the invention. The illustrated embodiments of the securement device are an anchor pad which is secured to the skin of a patient. Further, the illustrated embodiments are shown with either one or both of an illustrative example of a medical article, such as a connector fitting and/or a medical line, and an illustrative example of a retainer configured to secure the medical article relative to the anchor pad.

The illustration of the anchor pad in this context is not intended to limit the disclosed aspects and features of the invention to the specified embodiments or to usage with a medical article or retainer. Those of skill in the art will recognize that the disclosed aspects and features of the invention are not limited to use with any particular embodiment of a securement device, medical article, or retainer. For example, the securement device may be an anchor pad which includes one or more of the inventive aspects and features herein described can be designed for use with or without a variety of medical articles and/or retainers.

In order to reduce problems associated with long-term placement of intravenous catheters, including infections, extravasation resulting from patient movement, and clogged or collapsed veins, healthcare providers sometimes seek to periodically replace and/or re-site a medical line after a certain length of dwell time. To indicate dwell time, healthcare providers typically write the date and/or time that the line was inserted on a sticker or a piece of tape which is placed on the patient's skin near the insertion site. If the healthcare provider omits this step, or writes down an incorrect date, it becomes far more difficult to determine the actual dwell time.

Embodiments of the present invention advantageously provide a timer. The timer is disposed on a securement device such as an anchor pad or retainer. The securement device may stabilize a medical article or line relative to the skin of a patient. The timer can measure elapsed time. The timer can be activated by a user or by other means such as by contact with the skin of the patient. The timer indicates a time-based characteristic of the medical article or line, such as, for example, the length of time the medical article or line has been in place on the patient.

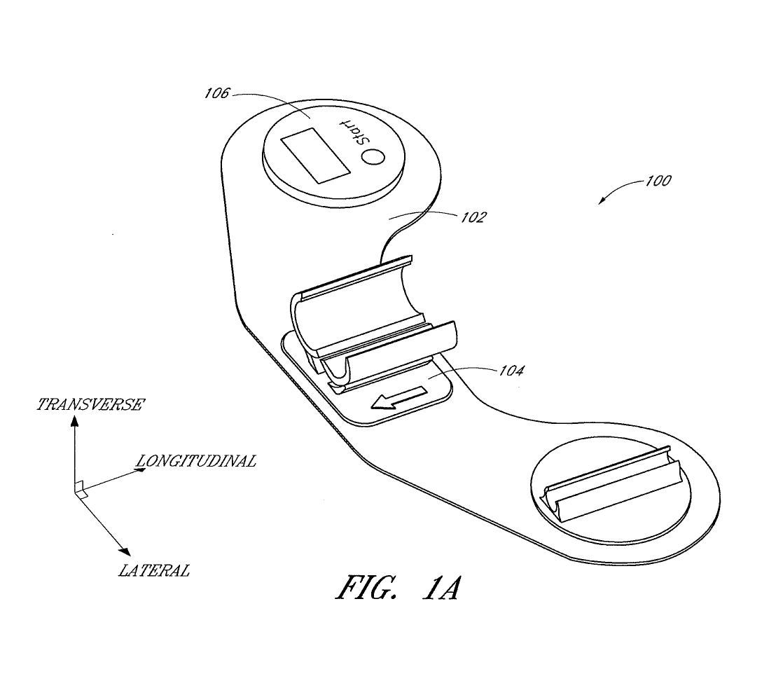

In certain embodiments, the securement device is an anchor pad, a retainer, or a combination of an anchor pad and retainer such as what is illustrated in FIG. 1A. Of course the securement device is not limited to the illustrated embodiments and may be in the form of other securement devices besides retainers and anchor pads.

The retainer of the securement device of FIG. 1A is disposed on the anchor pad. The retainer can be configured to inhibit movement of the medical line (and/or movement of a medical article connected to the medical line) in longitudinal, lateral, and/or transverse directions when the medical line is placed within the retainer.

The timer can be flexible or rigid, and can be disposed directly on the anchor pad, on a flexible or rigid substrate disposed on the anchor pad, or on a retainer supported by the anchor pad. By incorporating a timer as part of a securement device such as an anchor pad, embodiments of the invention provide an easy-to-use and reliable visual indicator of elapsed time since placement of the medical article or line.

With reference now to FIG. 1A, a securement system 100 is illustrated that includes an anchor pad 102 supporting a retainer 104 and a timer 106. FIG. 1B shows the securement system 100 securing an exemplary medical article 108, including an exemplary medical line 110. In the embodiment illustrated in FIGS. 1A and 1B, the timer 106 is spaced apart from the retainer 104 on the anchor pad 102, away from an insertion site and away from the path of the medical line 110. In other embodiments, the timer 106 can be disposed closer to the retainer 104, or on a portion of the retainer 104. The timer 106 can be activated by a healthcare provider at generally the same time as the medical line 110 is inserted and secured to the patient (or just prior to or just after insertion) and provide a visual indication of the length of time elapsed or period since insertion.

The period between indications can be fixed or variable. For example, the timer can provide an indication after a first time period and then provided a second indication after a second time period. The first and second time periods may have the same or different durations. The first indication may be the same or different than the second indication. For example, the timer can provided an audible indication after the first time period and a visual indication after the second time period. Thus, the timer 106 can be used to signal when the medical line should be replaced and/or re-sited.

The timer 106 can be user activated and provide a visual indication of elapsed time. For example, the time 106 can be a battery-operated timer or a chemically-active timer. Embodiments of a chemically active timer can change color or provide another visual response when exposed to air or a selected chemical for a given length of time.

The timer 106 can be flexible or rigid, and can be disposed directly on the anchor pad 102, on a substrate disposed on the anchor pad 102, or on a portion of a retainer supported by the anchor pad 102. The timer 106 may provide, in addition to or instead of a visual response, an audible indication of a given length of time.

The anchor pad 102 can have a variety of shapes, and can comprise a single unitary pad or a plurality of separated sections. The anchor pad 102 desirably comprises a laminate structure with an upper plastic, paper or foam layer (e.g., closed-cell polyethylene foam) and a lower adhesive layer. The lower adhesive layer constitutes a lower surface of the anchor pad. The lower surface desirably is a medical-grade adhesive and can be either diaphoretic or nondiaphoretic, depending upon the particular application. Such foam with an adhesive layer is available commercially from Avery Dennison of Painsville, Ohio.

In other variations, a hydrocolloid adhesive or zinc oxide-based adhesive can advantageously be used upon the anchor pad 102 for attaching the anchor pad to the skin of the patient. The hydrocolloid or zinc oxide-based adhesive can be used either alone or in combination with another medical grade adhesive (e.g., in combination with the adhesive available from Avery Dennison). Hydrocolloid and zinc oxide-based adhesives have less of a tendency to excoriate the skin of a patient when removed. This can be particularly important for patients whose skin is more sensitive or fragile, such as neonates and those with a collagen deficiency or other skin related condition.

In another variation, the anchor pad 102 comprises a laminate structure with an upper woven layer and a lower adhesive layer. The upper layer can be polyester or other suitable polymer or textile materials. One particular suitable material is woven polyester available commercially under the name “Tricot” from Tyco. The lower adhesive layer constitutes the lower surface of the anchor pad 102. The lower surface desirably is a medical-grade adhesive and can be either diaphoretic or nondiaphoretic, depending upon the particular application.

A surface of the upper foam layer constitutes an upper surface of the anchor pad 102. The upper surface can be roughened by corona-treating the foam with a low electric charge. In a further variation, the anchor pad 102 can comprise an upper paper or other woven or nonwoven cloth or plastic layer in lieu of a roughened upper foam surface.

Although not illustrated, a removable paper or plastic release liner desirably covers the adhesive lower surface before use. The liner preferably resists tearing and desirably is divided into a plurality of pieces to ease attachment of the pad to a patient's skin.

The retainer 104 can include a generally rigid structure (at least in comparison to foam or tape), but can also include structure and/or materials that lend some flexibility to part or all of the retainer. Suitably rigid but flexible materials include, for example, but without limitation: plastics, polymers or composites such as polypropylene, polyethylene, polycarbonate, polyvinylchloride, acrylonitrile butadiene styrene, nylon, olefin, acrylic, polyester, as well as moldable silicon, thermoplastic urethane, thermoplastic elastomers, thermoset plastics and the like. However, other materials can be utilized.

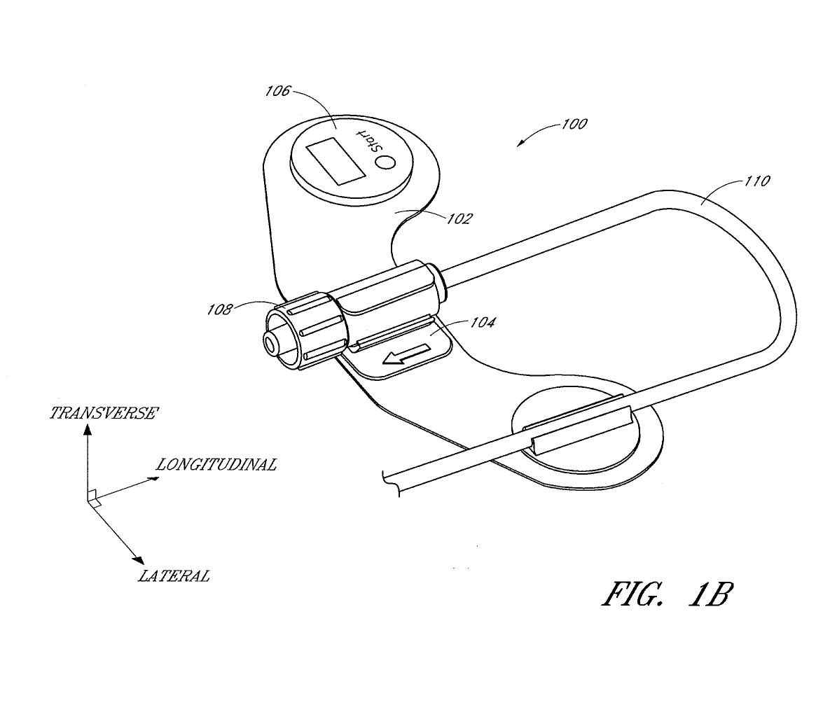

FIGS. 2A through 2C illustrate a portion of a securement device in the form of an anchor pad 200. The anchor pad 200 includes a chemically-active timer 202. The timer 202 can visually indicate the length of time elapsed in response to exposure to the ambient environment. To that end, the timer 202 can include a chemically-active region 204 and a removable, flexible protective layer 206 (FIG. 2A) configured to protect the active region 204 from the ambient environment until the layer 206 is removed by a healthcare provider (FIG. 2B). The chemically-active region 204 provides a visual indication of the length of time the region 204 has been exposed to a particular chemical, such as, for example, oxygen, air, etc. The protective layer 206 can be provided with a tacky or adhesive region or layer on its underside so as to form a seal against the active region 204 and prevent activation of the active region 204 until the protective layer 206 is removed. As can be seen in FIG. 2A, the protective layer 206 is sized slightly larger than the active region 204, and includes a pull tab 208 that extends beyond the tacky or adhesive region on the underside of the protective layer 206 to allow a healthcare provider to easily grip the tab 208 and remove the protective layer 206 to expose the active region 204 to the ambient environment at the appropriate time. The pull tab 208 can also extend beyond an edge of the anchor pad 200 to provide easier access to the pull tab 208.

The chemically-active region 204 can include two or more sub regions. The exposure sensitivity can be different for each sub region. For example, one or more of the sub regions can respond differently to chemical exposure. In certain embodiment, the one or more sub regions include pre-set graphical indicators, in order to indicate, with the desired specificity for the particular application, the length of time the region 204 has been exposed to the particular chemical.

In the embodiment illustrated in FIGS. 2A and 2B, once exposed, the chemically-active region 204 is configured to react with oxygen in the ambient environment and undergo a gradual change in color with continued exposure to the ambient environment. The active region 204 in the embodiment illustrated in FIGS. 2A and 2B changes color starting in a central portion 210 of the active region 204, and progresses outward as time passes.

As illustrated in FIG. 2B, the active region 204 also includes static graphic markers to indicate the meaning of a particular extent of color change. The inner ring 214 and the marker “2” together indicate that, when the region inside the inner ring 214 has completely changed color, approximately 2 days have passed since the active region 204 was exposed. The outer ring 216 and the marker “4” together indicate that, when the region inside the outer ring 216 has completely changed color, approximately 4 days have passed since the active region 204 was exposed, thus signaling that the medical line should be replaced or re-sited.

Although illustrated with markers to indicate the passage of 2 and 4 days since activation, timers according to embodiments can be configured to change color and/or provide a visual indicator of elapsed time according to any other desired schedule. Further, although illustrated with an active region that changes color from the inner portion of the region toward the outer portion, a reverse configuration is also possible, in which the active region is configured to change color from an outer region toward an inner region. In some embodiments, the entire chemically-active region 204 can be configured to change color at the same time, such that a complete color change of the active region 204 (for example from grey to red, or vice-versa) signals that the medical line should be replaced or re-sited. In some embodiments, the active region can include a fadable ink which is configured to fade upon exposure to air for a certain length of time. Embodiments can also include active regions that change color in any other desired manner, as well as any desired visual markers that cooperate with the active region to indicate when a particular length of time has passed since exposure. In some embodiments, the active region can include a static visual marker which is not visible immediately upon exposure, but which becomes visible as the active region fades or changes in color.

As illustrated in FIG. 2C, the chemically active region 204 can be disposed on a substrate 218, which can be secured in position on the anchor pad 200 by an adhesive layer. Although illustrated as a single layer, the active region 204 can comprise one or more thin films or layers, the particular composition of which can be selected for the desired rate and/or quality of color change. The active region 204 can be formed on the substrate by screen printing or any other suitable method.

In some embodiments, the substrate 218 is flexible so as to conform to the shape of the user's body along with the anchor pad 200. In one embodiment, the substrate 218 comprises paper. In other embodiments, the substrate 218 can comprise a rigid material, such as, for example, plastic. In some embodiments, the substrate 218 can be disposed directly over the anchor pad 200, while in other embodiments, the substrate 218 can be disposed within an indentation, well, or hole formed in the anchor pad 200. In some embodiments, the substrate 218 can be a lower layer of the anchor pad 200.

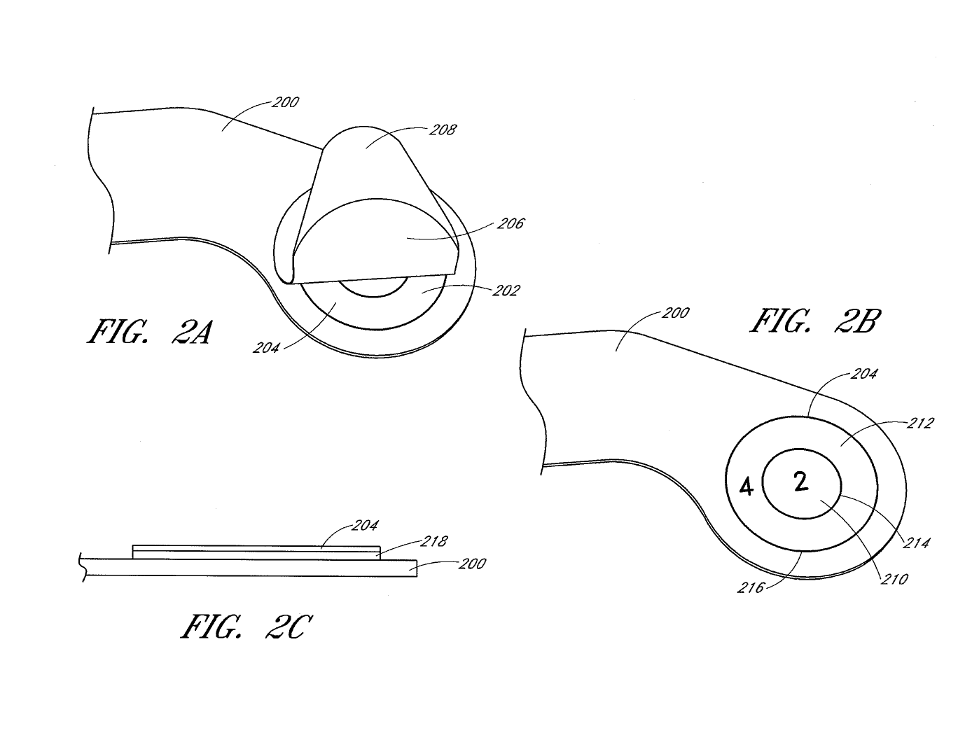

With reference now to FIG. 3, a portion of an anchor pad 300 according to another embodiment is illustrated. The anchor pad 300 includes a timer 302, which can be a battery-operated digital timer. The timer 302 can include a user-activated start button 304 and a display region 306 configured to display the length of time elapsed since the user activated the start button 304. In some embodiments, the start button 304 can comprise a button protruding from the upper surface of the timer 302. The button 304 can be activated by sufficient downward pressure or contact by the user. In other embodiments, the start button is recessed below the upper surface of the timer 302, requiring insertion of a stylus or other object in order to activate the timer 302. Such an arrangement may reduce the likelihood of premature or accidental activation of the timer 302 by the healthcare provider or patient. The anchor pad 300 can also include a removable protective layer or cap over the timer 302 which is configured to prevent activation of the start button 304 before the intended time. In some embodiments, a stop button can also be provided and configured to allow a healthcare provider to stop the timer from running in the case of accidental activation.

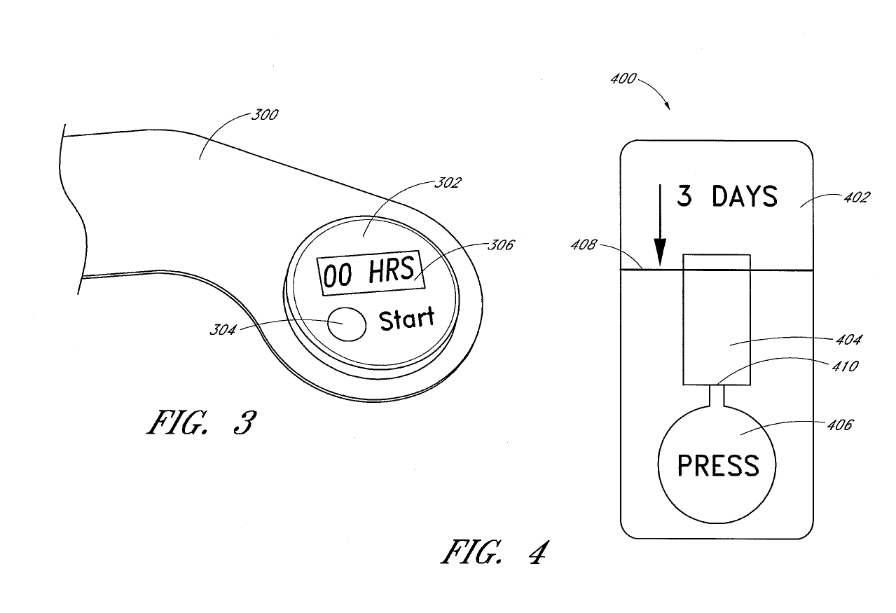

FIG. 4 shows a timer 400 according to another embodiment. The timer 400 includes a substantially rigid substrate 402, a chemically active region 404, and a chemical reservoir 406, and a graphic marker 408. The chemically active region 404 and the chemical reservoir 406 can be sealed from the outside environment by a protective layer that covers all or part of the upper surface of the timer 400. The chemically active region 404 and the chemical reservoir 406 can be isolated or sealed off from one another by a barrier 410. To activate the timer 400, the healthcare provider breaks the barrier 410. For example, the healthcare provider can apply downward pressure to the protective layer overlying the chemical reservoir 406. The pressure breaks the barrier 410 between the chemical reservoir 406 and the chemically active region 404 allowing the chemical in the reservoir 406 to come in contact with the active region 404. The chemically active region 404 gradually changes color in response to exposure to the chemical in the reservoir 406. For example, the color changes starting from the barrier 410 and moving toward the graphical marker 408. When the color change reaches the graphical marker 408, the healthcare provider is signaled to replace and/or re-site the medical line. In some embodiments, the timer 400 can be disposed on an anchor pad, such as the anchor pads 102, 200, and 300 illustrated in FIGS. 1 through 3.

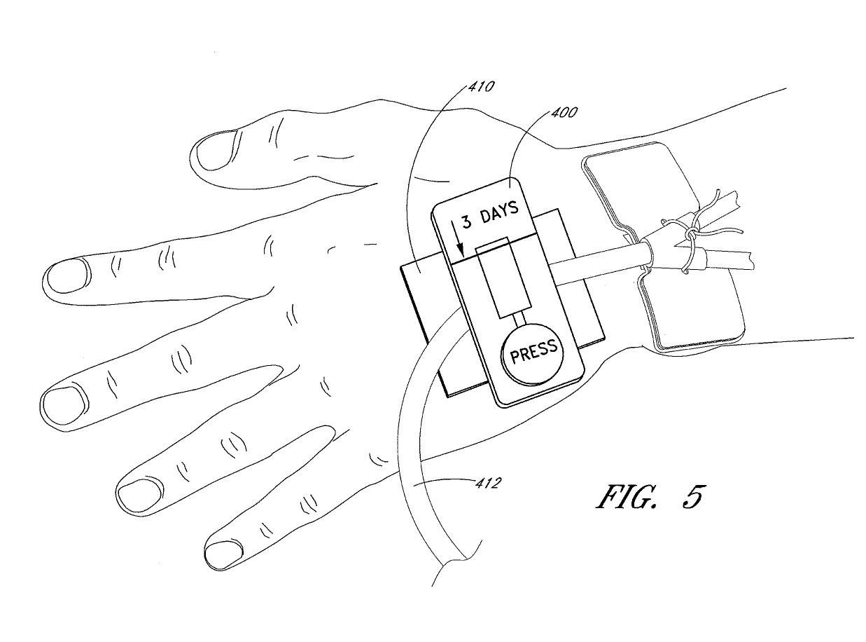

In other embodiments, the timer 400 is disposed on an upper surface of a retainer which itself is located on anchor pad 414. In such an embodiment, the timer 400 is disposed on the retainer. FIG. 5, for example, illustrates a timer 400 located over both the anchor pad 414 and the retainer (not shown). The retainer is disposed between the timer 400 and the anchor pad 414 and secures a medical line 412 to the skin of a patient.

The following method of use will be with reference principally to FIGS. 4 and 5, and will be in the context of starting an intravenous line. This discussion of one embodiment of a method of use is meant to augment the description of other embodiments above and both should be read together.

In an embodiment of the invention, a healthcare provider can indicate dwell time of an intravenous line by inserting an end of a catheter or medical line, such as the medical line 412, into a patient's vasculature at an insertion site using known procedures. For example, prior to insertion, a needle, stylus, or trocar can be slidably received within a portion or portions of the medical line and then removed after the medical line has been inserted into the patient's vasculature. In some embodiments, the medical line is primed with sterile fluid to ensure the flow of fluid through the system.

After inserting the medical line, the healthcare provider can connect the medical line to a fluid supply line. With the medical line inserted and connected to the fluid supply line, the healthcare provider applies the anchor pad 410 to the skin of the patient, near the insertion site. The healthcare provider starts the timer 400 on the anchor pad 410 by activating the timer 400. In the embodiment illustrated in FIGS. 4 and 5, for example, the healthcare provider activates the timer 400 by applying downward pressure to the protective layer overlying the chemical reservoir 406, thereby breaking the barrier 410 between the chemical reservoir 406 and the chemically active region 404 and bringing the chemical in the reservoir 406 in contact with the active region 404. In some embodiments, the healthcare provider can also use the retainer (not shown) on the anchor pad to secure the medical line 412 (or a portion thereof) to the patient's skin.

In embodiments of the invention, an in-use timer can form a component of a catheterization or securement system that includes one or more medical articles, such as connector fittings, catheters, hubs, catheter adaptors, fluid supply lines, or other articles suitable for securement via one or more anchor pad and/or retainer.

The various embodiments of securement devices and techniques described above thus provide a number of ways to provide a reliable indication of the length of time elapsed since initial placement of a medical line into a patient's body. Embodiments of the invention can be adapted for use with a variety of securement devices, for example, anchor pads and retainers. Further, embodiments of the invention can be adapted for use with a variety of medical articles. The medical article can be a single medical article or a combination of one or more medical articles. Such medical articles can be or include, for example, but without limitation, connector fittings, catheters, catheter hubs, catheter adaptors, fluid supply lines, or other similar articles. In addition, the techniques described may be broadly applied for use with a variety of medical lines and medical procedures.

Of course, it is to be understood that not necessarily all such objectives or advantages may be achieved in accordance with any particular embodiment using the systems described herein. Thus, for example, those skilled in the art will recognize that the systems may be developed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Although these techniques and systems have been disclosed in the context of certain embodiments and examples, it will be understood by those skilled in the art that these techniques and systems may be extended beyond the specifically disclosed embodiments to other embodiments and/or uses and obvious modifications and equivalents thereof. Additionally, it is contemplated that various aspects and features of the invention described can be practiced separately, combined together, or substituted for one another, and that a variety of combination and subcombinations of the features and aspects can be made and still fall within the scope of the invention. Thus, it is intended that the scope of the systems disclosed herein disclosed should not be limited by the particular disclosed embodiments described above.

What is claimed is:

1. An anchor pad for securing to a patient, the anchor pad comprising a timer.

2. The anchor pad of claim 1, wherein the timer is user activated.

3. The anchor pad of claim 1, wherein the timer provides an indication of elapsed time.

4. The anchor pad of claim 3, wherein the indication provided by the timer is visual.

5. The anchor pad of claim 3, wherein the indication provided by the timer is audible.

6. The anchor pad of claim 1 further comprising a display.

7. The anchor pad of claim 1 further comprising a protective layer covering at least a portion of the timer.

8. The anchor pad of claim 1 further comprising a chemical reservoir and a chemically active region.

9. The anchor pad of claim 8 further comprising a breakable barrier disposed between the chemical reservoir and the chemically active region.

10. A securement system for securing a medical article to the skin of a patient, the securement system comprising:
an anchor pad having a lower surface, at least a portion of the lower surface being covered by an adhesive;
a retainer supported by the anchor pad, the retainer being configured to receive at least a portion of the medical article; and
a timer.

11. The securement system of claim 10, wherein the timer is disposed on the retainer.

12. The securement system of claim 10, wherein the timer indicates elapsed time.

13. The securement system of claim 10, wherein the timer is flexible.

14. The securement system of claim 10, wherein the timer is rigid.

15. The securement system of claim 10, wherein the timer is disposed on a substrate, the substrate being disposed on the anchor pad.

16. A method of indicating dwell time of a medical line in a patient, the method comprising:
inserting the medical line at an insertion site in the skin of the patient;
securing an anchor pad to the skin of the patient near the insertion site, the anchor pad comprising a timer configured to provide an indication of elapsed time; and
activating the timer.

17. The method of claim 16 further comprising securing at least a portion of the medical line using a retainer disposed on the anchor pad.

18. The method of claim 16, wherein the timer is configured to provide an audible indication of elapsed time.

19. The method of claim 16, wherein the timer is configured to provide a visible indication of elapsed time.

20. The method of claim 19, wherein the timer comprises a chemical reservoir separated from a chemically active region by a breakable barrier.
